(19) United States
(12) Patent Application Publication
(10) Pub. No.: US 2011/0184503 A1
(43) Pub. Date: Jul. 28, 2011

(54) METHOD OF MAKING 3-DIMENSIONAL NEURAL PROBES HAVING ELECTRICAL AND CHEMICAL INTERFACES

(76) Inventors: Yong Xu, Troy, MI (US); Yuefa Li, Detroit, MI (US)

(21) Appl. No.: 12/737,126

(22) PCT Filed: Jun. 16, 2009
(86) PCT No.: PCT/US2009/003631

§ 371 Date: Feb. 22, 2011

Related U.S. Application Data
(60) Provisional application No. 61/132,199, filed on Jun. 16, 2008.
(60) Provisional application No. 61/194,940, filed on Oct. 1, 2008.

(51) Int. Cl. A61N 1/05; C23F 1/00

(52) U.S. Cl. 607/116; 216/11

(57) ABSTRACT

A method of fabricating a three-dimensional neural probe includes the steps of: growing thermal oxide layer; depositing a layer of Au/Cr on the thermal oxide layer; patterning the layer of Au/Cr; depositing a layer of parylene C; etching the thermal oxide layer to release a plurality of islands; and folding the islands onto one another in stacked relation. The layer of Au/Cr is formed by an evaporation process, and the layer of parylene C is deposited to a thickness of approximately 8 μm. DRIE is used to perform the etching, and HF is used to remove the thermal oxide. A spacer is disposed intermediate of two of the islands.

### RELATIONSHIP TO OTHER APPLICATIONS

This application is related to, and claims the benefit of the filing dates of, Provisional Patent Application Ser. No. 61/132,199, filed on Jun. 16, 2008; and Provisional Patent Application Ser. No. 61/194,940 filed on Oct. 1, 2008. The disclosures of these provisional patent applications are incorporated herein by reference.

### BACKGROUND OF THE INVENTION

1. Field of the Invention

This invention relates generally to neural probes, and more particularly, to a method of making three-dimensional neural probes whereby yield is increased and the cost of three-dimensional neural probes is significantly decreased.

2. Description of the Related Art

Three-dimensional neural probes are highly desirable for various forms of neurological research and for medical applications. For example, such probes are useful for the restoration of sight to blind people, or the restoration of motor and sensory function to paralyzed patients. A large variety of other neurological disorders, such as Parkinson's disease, might also be treated with three-dimensional neural probes.

Several research groups have developed three-dimensional neural probes based on different technologies. The two well-known examples are Utah electrodes and Michigan probes. Some significant advantages of the three-dimensional neural probes produced in accordance with the inventive method herein disclosed include simplicity and design flexibility. Compared with known Michigan probes, the yield is higher and the cost is much lower, thereby enabling broader dissemination of the three-dimensional neural probes. Compared with the known Utah electrodes, the technology herein described allows: (1) longer probes; (2) the integration of multiple electrode sites on one probe, so as to enable simultaneous detection of neural activities at different depths; and (3) the integration of integrated circuits, thereby simplifying the wiring and increasing the signal-to-noise ratio. It is another important advantage of the method of the present invention that microchannels can be integrated for delivery of medications.

3D neural probes are especially desirable because of the 3D nature of nervous systems. Several research groups have already developed 3D neural probes based on different technologies. Compared with Michigan probes, the new technology herein described has simpler fabrication/assembly process and thus the cost is much lower. Compared with the Utah electrodes, the new technology enables longer probes and the integration of multiple electrode sites on one probe for high-density recording/stimulating, and enables the integration of microfluidic channels into 3D probes. These microchannels can deliver neurotransmitters for chemical stimulation/regulation of neurons, and also deliver drugs to reduce tissue reaction/inflammation, prevent biofouling, promote neuron growth, or treat certain diseases.

It is, therefore, an object of this invention to provide a method of manufacturing three-dimensional neural probes that are simpler and less expensive to produce than known three-dimensional neural probes.

It is another object of this invention to provide a method of manufacturing three-dimensional neural probes that affords improved yield over known manufacturing methods.

It is also an object of this invention to provide a method of manufacturing three-dimensional neural probes that are longer than conventional three-dimensional neural probes.

It is a further object of this invention to provide a method of manufacturing three-dimensional neural probes that enables the production of multiple electrode sites integrated on one probe.

It is additionally an object of this invention to provide a method of manufacturing three-dimensional neural probes that enables the integration of integrated circuits, thereby enabling simplification of the wiring and an increase the signal-to-noise ratio, over known three-dimensional neural probe arrangements.

It is an additional object of the invention to provide a method of manufacturing three-dimensional neural probes that enables the integration of microfluidic channels that facilitate the delivery of medications.

### SUMMARY OF THE INVENTION

The foregoing and other objects are achieved by this invention which provides a method of fabricating a three-dimensional neural probe. The method includes the steps of:

growing thermal oxide layer;
depositing a layer of Au/Cr on the thermal oxide layer;

patterning the layer of Au/Cr;

depositing a layer of parylene C;

etching the thermal oxide layer to release a plurality of islands; and

folding the islands onto one another in stacked relation.

In one embodiment of the invention, the layer of Au/Cr is formed by an evaporation process.

In a further embodiment, the layer of parylene C is deposited to a thickness of approximately 8 μm.

In a still further embodiment, the step of etching includes the step of using deep reactive ion etching (“DRIE”). HF is used in some embodiments to remove the thermal oxide.

The step of folding the islands onto one another in stacked relation includes, in some embodiments, the further step of providing a spacer intermediate of two of the islands.

In accordance with an advantageous embodiment of the invention, there is provided a method of making 3D penetrating neural probes with combined electrical and chemical interfaces. The electrical interface is provided by metal electrodes and the chemical interface is provided by micro-channels. One highly desirable feature of the novel neural probes is the integration of microfluidic channels for the delivery of chemicals. These micro-channels enable delivery of neurotransmitters for chemical stimulation/regulation of neurons, and are also useful to effect delivery of drugs that reduce tissue reaction/inflammation, prevent biofouling, promote neuron growth, or treat certain diseases. Alternatively, the micro-channels extract extracellular fluid for chemical analysis. There is not present in the art an adequate method of integrating microfluidic channels into 3D neural probes, as it is very challenging to route the micro-channels in 3D space.

The novel 3-D neural probes having chemical delivery capability as herein described are highly desirable for various neurological researches and medical applications. For example, these probes can be used for the restoration of sight of blind people or the motor and sensory function of paralyzed patients. Many other neurological disorders such as Parkinson's disease might be treated as well. In some embodiments, the chemicals are driven by external syringes. However, on-chip micro-pumps can be integrated with the 3-D neural probes.

In accordance with a further method aspect of the invention, there is provided a method of fabricating a three-dimensional neural probe. The method is provided with the steps of:

growing a thermal oxide layer;

etching the thermal oxide layer to release an island; and

forming a microchannel.

In one embodiment of this further method aspect of the invention, the step of forming a microchannel includes the steps of:

depositing a layer of a parylene C on a silicon substrate;

patterning the layer of a parylene C;

etching the silicon substrate to form a microchannel; and

sealing the microchannel formed in the step of etching the silicon substrate.

In an advantageous embodiment, the step of etching the silicon substrate to form a microchannel includes the step of etching with XeF₂. Also, the step of sealing the microchannel includes the step of depositing a further layer of parylene C. In some embodiments, the step of depositing a further layer of parylene C includes the further step of lining the microchannel formed in the silicon substrate with parylene C.

There is additionally provided in some embodiments the step of removing at least a portion of the silicon substrate. Such removal of the silicon substrate is effected in some embodiments by the process of deep reactive ion etching.

In a highly advantageous embodiment, there is further provided the step of etching the thermal oxide layer to release a further island. In accordance with the invention, the island and the further island are folded onto one another in stacked relation.

In accordance with an apparatus aspect of the invention, there is provided a three-dimensional neural probe arrangement having a first island and an electrical probe formed on the first island. A microchannel is arranged to extend from the first island.

In one embodiment of this apparatus aspect of the invention, there is further provided a second island. A flexible interconnection arrangement couples the first and second islands to each other, wherein when folded the first and second islands are disposed in stacked relation to one another.

In some embodiments, there is further provided a spacer interposed between the first and second islands. Also, some embodiments of the invention are provided a further electrical probe formed on the second island.

In a highly advantageous embodiment, the flexible interconnection arrangement is formed of parylene C.

### BRIEF DESCRIPTION OF THE DRAWING

Comprehension of the invention is facilitated by reading the following detailed description, in conjunction with the annexed drawing, in which:

FIG. 1 is a simplified schematic plan representation of a planar device constructed in accordance with the principles of the invention and having, in this specific illustrative embodiment of the invention, three silicon islands before folding;

FIGS. 2(a) and 2(b) are simplified schematic cross-sectional representations of the planar device of FIG. 1 that is useful to illustrate the method of assembling the three-dimensional neural probe;

FIGS. 3(a), 3(b), and 3(c) illustrate respective steps in the process of manufacturing a three-dimensional neural probe system;

FIG. 4 is a perspective representation of a fabricated specific illustrative embodiment of the invention having three islands;

FIG. 5 is a scanning electron microscope (SEM) image of a three-probe specific illustrative embodiment of the invention;

FIG. 6 is a perspective simplified schematic representation of a fabricated specific illustrative embodiment of a three-dimensional neural probe;

FIG. 7 is a perspective SEM image of the specific illustrative embodiment of the invention that is represented schematically in FIG. 6;

FIG. 8 is a perspective representation showing the details of folded gold traces between two of the islands;

FIG. 9 is a simplified schematic representation of a testing scheme for a folded three-island embodiment of the invention, wherein a square wave with 1 V amplitude is applied between electrode 1 and 5, electrode 5 serving as ground;

FIG. 10 is a graphical representation of the voltage obtained across electrodes 4 and 5 of the arrangement of FIG. 9;

FIGS. 11(a), 11(b), 11(c), and 11(d) are simplified schematic representations that are useful to illustrate a specific illustrative embodiment of a fabrication process of microchannels, wherein FIG. 11(a) represents the depositing and patterning of a parylene C layer; FIG. 11(b) represents etching with XeF₂; FIG. 11(c) illustrates the sealing and forming of a channel by depositing another parylene C layer; and FIG. 11(d) illustrates the removal of silicon by backside DRIE;

FIG. 12 is a scanning electron microscope (“SEM”) image of a microchannel before sealing;

FIG. 13 is a cross sectional representation of microchannel formed in accordance with the principles of the invention;

FIG. 14 is an illustration of a device constructed in accordance with the principles of the invention and having two silicon islands and two integrated microchannels;

FIG. 15 is a SEM image of the backside of a bent parylene connection layer between the two islands of the embodiment of FIG. 14; and

FIG. 16 is a representation of a liquid droplet that has emerged from the orifice of the microchannel at the probe tip.

### DETAILED DESCRIPTION

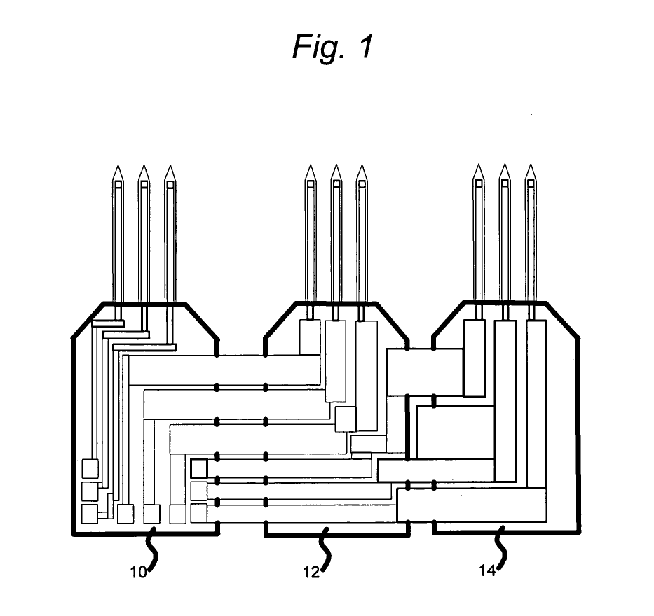

FIG. 1 is a simplified schematic plan representation of a planar device constructed in accordance with the principles of the invention and having, in this specific illustrative embodiment of the invention, three silicon islands 10, 12, and 14, before folding, as will be discussed below. It is to be understood that the number of islands is not limited to three, as in the specific illustrative embodiment of the invention shown and described herein.

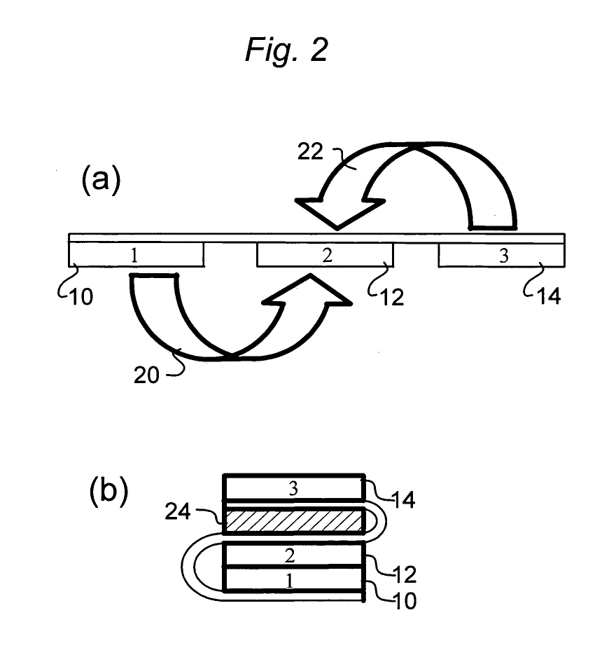

FIGS. 2(a) and 2(b) are simplified schematic cross-sectional representations of the planar device of FIG. 1 that is useful to illustrate the method of assembling the three-dimensional neural probe. Elements of structure that have previously been discussed are similarly designated. As shown in FIG. 2(a), islands 10, 12, and 14 are initially formed coplanar, and subsequently are folded in the directions of arrows 20 and 22.

FIG. 2(b) is a simplified schematic representation that illustrates the embodiment of FIGS. 1 and 2(a) in a folded condition. As shown, island 12 is arranged to overlie island 10. Island 14 overlies island 12, and there is interposed therebetween a spacer 24.

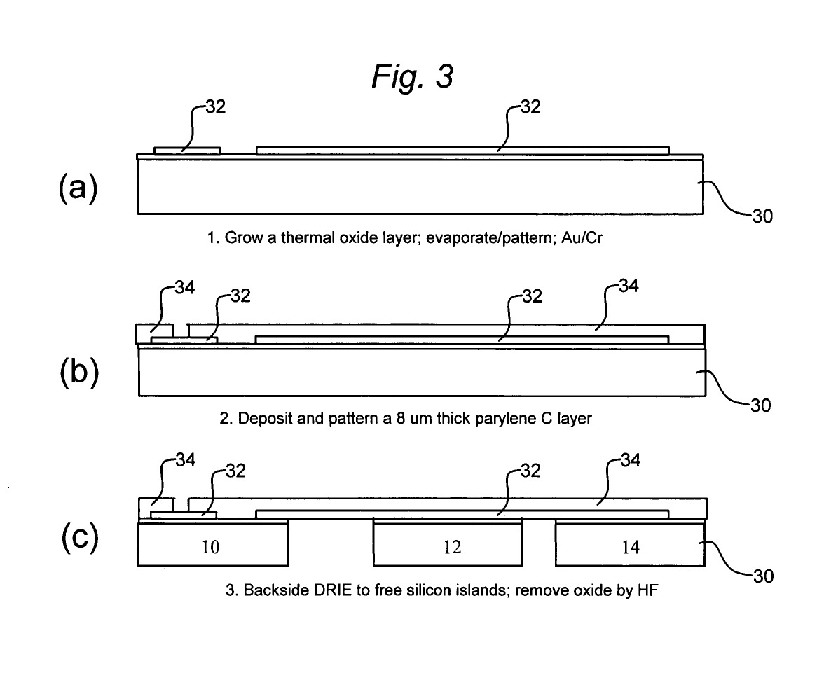

FIGS. 3(a), 3(b), and 3(c) illustrate respective steps in the process of manufacturing a three-dimensional neural probe system. In FIG. 3(a), a thermal oxide layer 30 is shown to have been grown, and a layer 32, illustratively of Au/Cr is deposited, illustratively by an evaporation process, and patterned thereon. In FIG. 3(b), there is shown to be deposited and patterned an 8 μm thick parylene C layer 34. In FIG. 3(c), elements of structure that have previously been discussed are similarly designated. This figure shows the backside deep reactive ion etching (DRIE) to free the silicon islands, and the thermal oxide has been removed by HF.

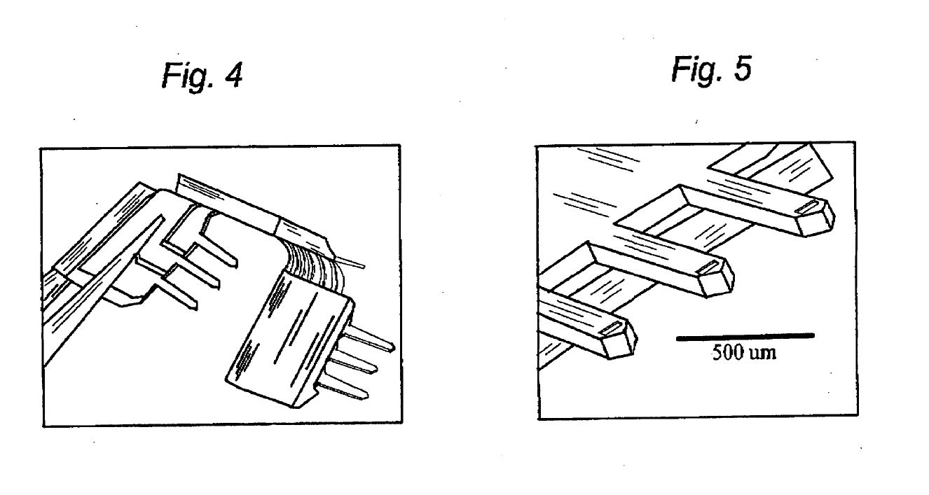

FIG. 4 is a perspective representation of a fabricated specific illustrative embodiment of the invention having three islands.

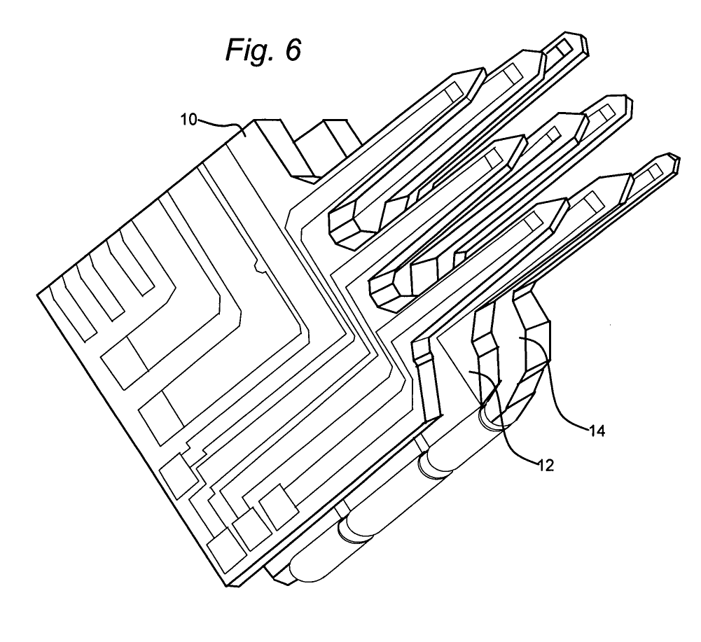

FIG. 5 is a scanning electron microscope (SEM) image of a three-probe specific illustrative embodiment of the invention.

FIG. 6 is a perspective representation of a fabricated specific illustrative embodiment of a three-dimensional neural probe.

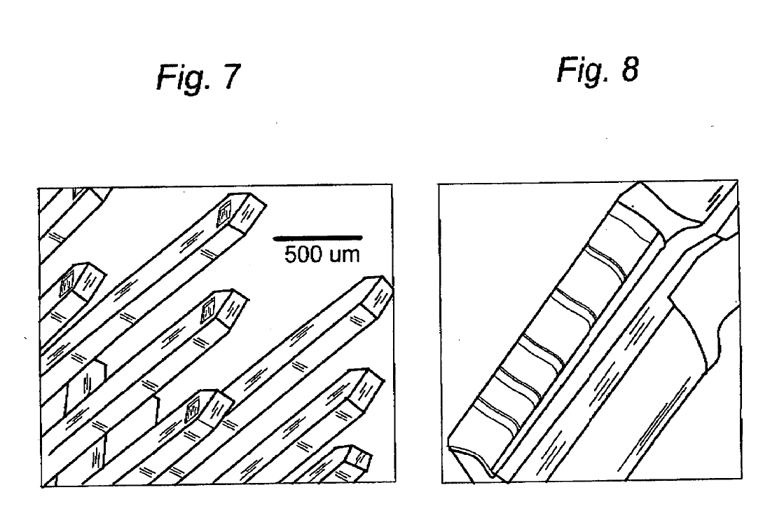

FIG. 7 is a perspective SEM image of the specific illustrative embodiment of the invention schematically represented in FIG. 6.

FIG. 8 is a perspective representation showing the details of folded gold traces between two of the islands.

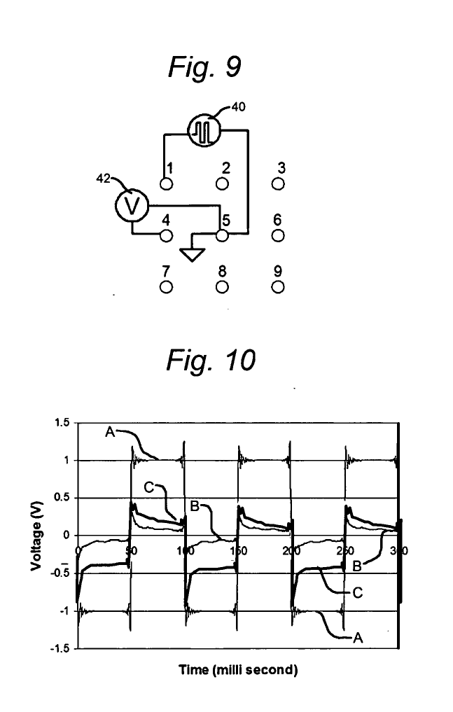

FIG. 9 is a simplified schematic representation of a testing scheme for a folded three-island embodiment of the invention, wherein a square wave with 1 V amplitude from a square wave generator 40 is applied between electrode 1 and 5, electrode 5 serving as ground. The voltage, as shown below in connection with FIG. 10, is monitored by a scope 42 across electrodes 4 and 5.

FIG. 10 is a graphical representation of the voltage obtained across electrodes 4 and 5 of the arrangement of FIG. 9. The experiment was performed in deionized (DI) water and repeated in 2×PBS solution. FIG. 10 illustrates the preliminary test results. A square waveform A represents the stimulating voltage provided by square wave generator 40. Waveform B represents the voltage recorded in across electrodes 4 and 5 in DI water, and waveform C represents the voltage recorded in PBS solution.

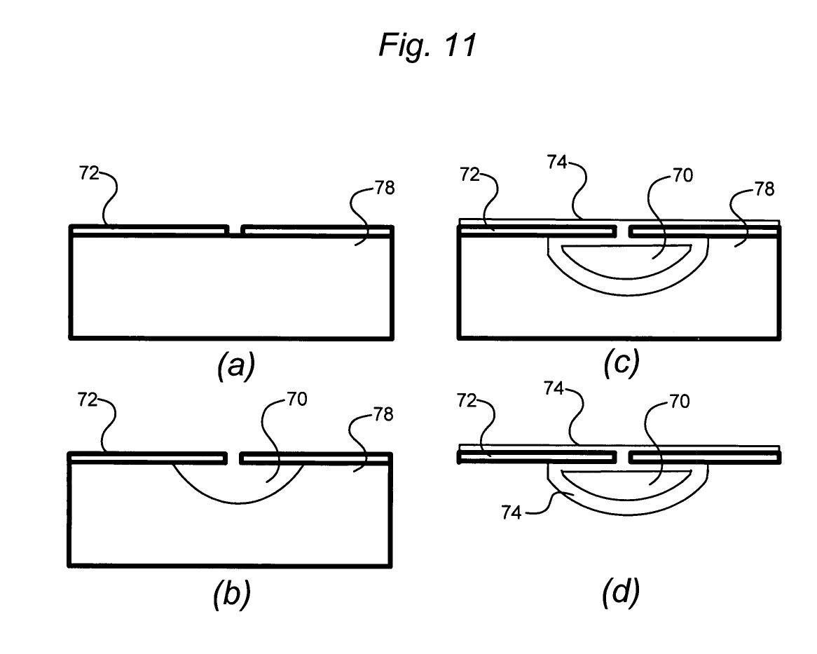

FIGS. 11(a), 11(b), 11(c), and 11(d) are simplified schematic representations that are useful to illustrate a specific illustrative embodiment of a fabrication process of a microchannel 70. More specifically, FIG. 11(a) represents the depositing and patterning of a parylene C layer 72 that has been deposited on a silicon substrate 78. FIG. 11(b) represents etching of silicon substrate 78 with XeF₂ to form microchannel 70. Elements of structure that have previously been discussed are similarly designated. FIG. 11(c) illustrates the sealing and forming of a channel by depositing another parylene C layer 74. As shown, the further layer of parylene C (74) additionally serves as a lining, for microchannel 70 that has been etched into silicon substrate 78. FIG. 11(d) illustrates the removal of silicon 78 by backside deep reactive ion etching (“DRIE”).

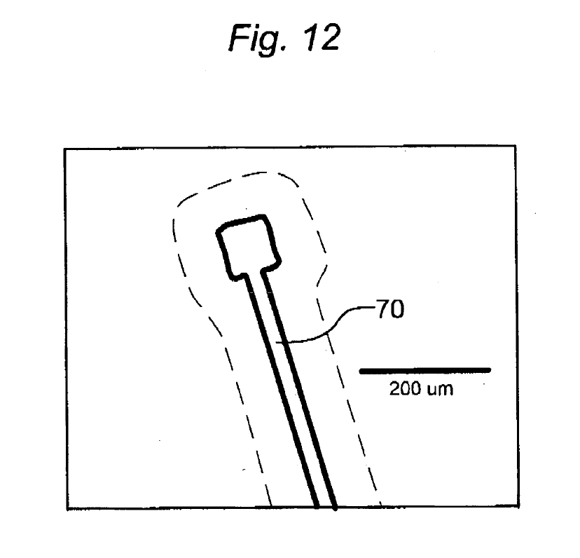

FIG. 12 is a scanning electron microscope (“SEM”) image of a specific illustrative embodiment of microchannel 70 before sealing. This figure shows a 200 μm reference length that serves to illustrate the approximate dimensions of this specific illustrative embodiment of the invention.

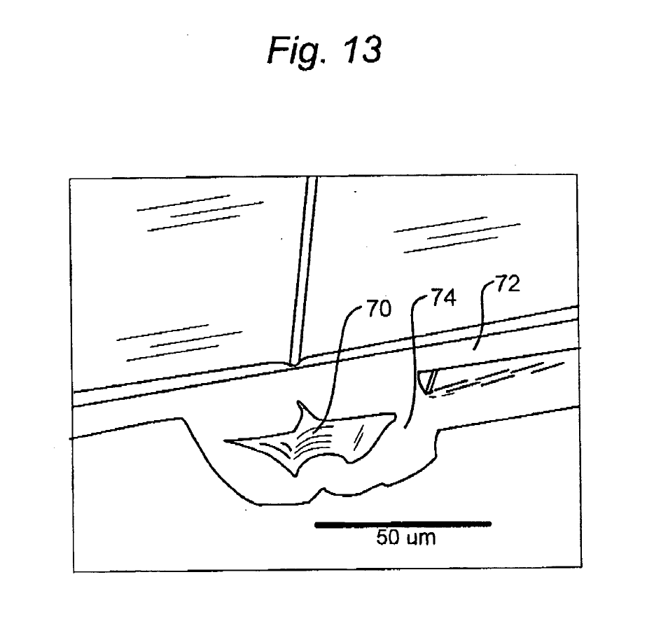

FIG. 13 is a cross sectional microscopic representation of microchannel 70 formed in accordance with the principles of the invention. Elements of structure that have previously been discussed are similarly designated. This figure shows a 50 μm reference length that serves to illustrate the approximate dimensions of this specific illustrative embodiment of the invention.

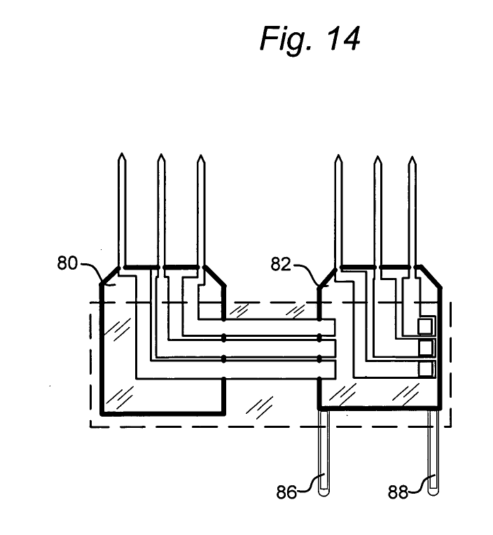

FIG. 14 is an illustration of a device constructed in accordance with the principles of the invention and having two silicon islands 80 and 82 and two integrated microchannels 86 and 88 extending from silicon island 82.

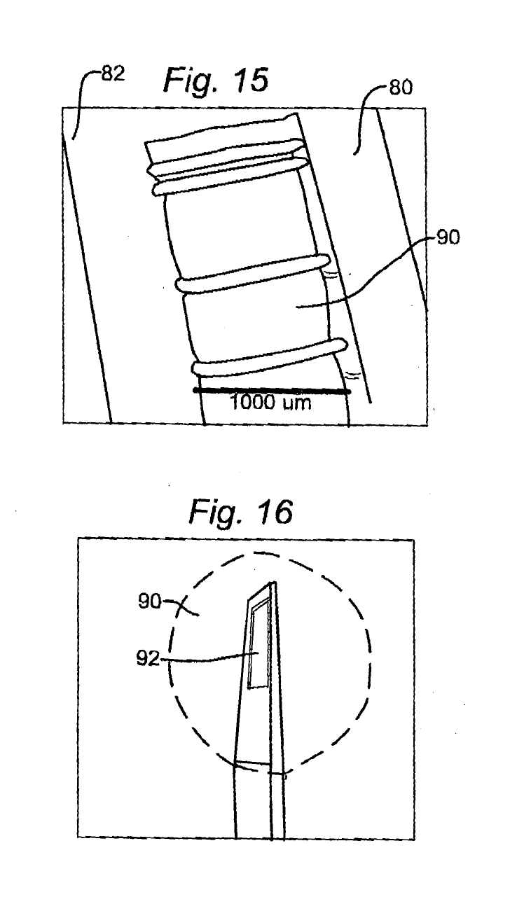

FIG. 15 is a SEM image of the backside of a bent parylene connection layer 90 between the two islands 80 and 82 of the embodiment of FIG. 14. This figure shows a 1000 μm reference length that serves to illustrate the approximate dimensions of this specific illustrative embodiment of the invention.

FIG. 16 is a representation of a liquid droplet 90 that has emerged from the orifice of a microchannel 92 at the probe tip.

Although the invention has been described in terms of specific embodiments and applications, persons skilled in the art may, in light of this teaching, generate additional embodiments without exceeding the scope or departing from the spirit of the invention described herein. Accordingly, it is to be understood that the drawing and description in this disclosure are proffered to facilitate comprehension of the invention, and should not be construed to limit the scope thereof.

What is claimed is:

1. A method of fabricating a three-dimensional neural probe, the method comprising the steps of
growing thermal oxide layer;
depositing a layer of Au/Cr on the thermal oxide layer;
patterning the layer of Au/Cr;
depositing a layer of parylene C;
etching the thermal oxide layer to release a plurality of islands; and
folding the islands onto one another in stacked relation.

2. The method of claim 1, wherein the layer of Au/Cr is formed by an evaporation process.

3. The method of claim 1, wherein the layer of parylene C is 8 μm thick.

4. The method of claim 1, wherein said step of etching comprises the step of using deep reactive ion etching (“DRIB”).

5. The method of claim 4, wherein said step of etching comprises the step of using HF to remove the thermal oxide.

6. The method of claim 1, wherein said step of folding the islands onto one another in stacked relation includes the further step of providing a spacer intermediate of two of the islands.

7. A method of fabricating a three-dimensional neural probe, the method comprising the steps of:
growing a thermal oxide layer;
etching the thermal oxide layer to release an island; and
forming a microchannel.

8. The method of claim 7, wherein said step of forming a microchannel comprises the steps of:
depositing a layer of a parylene C on a silicon substrate;
patterning the layer of a parylene C;
etching the silicon substrate to form a microchannel; and
sealing the microchannel formed in said step of etching the silicon substrate.

9. The method of claim 8, wherein said step of etching the silicon substrate to form a microchannel comprises the step of etching with XeF₂.

10. The method of claim 8, wherein said step of sealing the microchannel comprises the step of depositing a further layer of parylene C.

11. The method of claim 10, wherein said step of depositing a further layer of parylene C includes the further step of lining the microchannel formed in the silicon substrate with parylene C.

12. The method of claim 8, wherein there is further provided the step of removing at least a portion of the silicon substrate.

13. The method of claim 12, wherein said step of removing at least a portion of the silicon substrate is performed by the process of deep reactive ion etching.

14. The method of claim 7, wherein there is further provided the step of etching the thermal oxide layer to release a further island.

15. The method of claim 14, wherein there is further provided the step of folding the island and the further island onto one another in stacked relation.

16. A three-dimensional neural probe arrangement comprising:
a first island;
an electrical probe formed on said first island; and
a microchannel arranged to extend from said first island.

17. The three-dimensional neural probe arrangement of claim 16, wherein there is further provided;
a second island; and
a flexible interconnection arrangement for coupling said first and second islands to each other;
wherein when folded said first and second islands are disposed in stacked relation to one another.

18. The three-dimensional neural probe arrangement of claim 17, wherein there is further provided a spacer interposed between said first and second islands.

19. The three-dimensional neural probe arrangement of claim 17, wherein there is further provided a further electrical probe formed on said second island.

20. The three-dimensional neural probe arrangement of claim 17, wherein said flexible interconnection arrangement is formed of parylene C.
